(19) United States
(12) Patent Application Publication
(10) Pub. No.: US 2025/0069690 A1
(43) Pub. Date: Feb. 27, 2025

(54) MACHINE LEARNING-BASED SYSTEM USING LOW-N LEARNING TO ACCURATELY IDENTIFY VARIANTS FOR ENGINEER GENOME EDITING PROTEINS

(71) Applicants: The University of Hong Kong, Hong Kong (HK); Centre for Oncology and Immunology Limited, Hong Kong (HK)

(72) Inventors: Siu Lun WONG, Hong Kong (HK); Hoi Yee CHU, Hong Kong (HK)

(21) Appl. No.: 18/783,475

(22) Filed: Jul. 25, 2024

Related U.S. Application Data
(60) Provisional application No. 63/578,783, filed on Aug. 25, 2023.

(51) Int. Cl. G16B 20/20

(52) U.S. Cl. CPC G16B 20/20

(57) ABSTRACT

A machine learning-based top variant identification pipeline method and systems for engineering genome editing proteins are provided. The method includes steps of performing zero-shot predictions by a zero-shot predictor together with a clustered-based sampling (CS) or Top regime method with wild-type (WT) being added to training data for low-N training data preparation; performing machine learning-assisted directed evolution (MLDE) predictions based on the training data prepared; and performing multi-round sampling of the CS/MLDE predictions, in which the wild type is added in a first-round sampling of the multi-round sampling. The zero-shot predictor is based on an arDCA method, an EVmutation method, an EvoEF method or a MSA method. The performance of the zero-shot predictor is evaluated based on performance metrics including Spearman's correlation (rho) and/or top 5% overlaps (overlap_5).

### CROSS-REFERENCE TO RELATED APPLICATIONS

The present application claims the priority from the U.S. provisional patent application Ser. No. 63/578,783 filed Aug. 25, 2023, and the disclosure of which is incorporated herein by reference in its entirety.

### FIELD OF THE INVENTION

The present invention generally relates to at least the fields of machine learning and data analysis.

### BACKGROUND OF THE INVENTION

In recent years, the genome editing toolkit has been developed to include an expanding collection of CRISPR-Cas protein variants mainly derived from rational design, directed evolution, and combinatorial mutagenesis. Nevertheless, these methodologies either require extensive understanding of the protein structures involved or laborious selection and cloning for variant screening. Machine-learning based in-silico screen emerges to be an appealing method to search for high-performance genome editor variants, reducing up to 80% of experimental burden to save both time and resource in solving the Cas9 optimization problems 1.

Machine learning (ML)-guided methodologies which enable in-silico screens and foster protein engineering by increasing the speed of identifying valuable variants and the number of possible mutations were explored. Traditionally, many protein optimization tasks were confined by the wet-lab screening capacity for evaluating beneficial mutation combinations. To address the scalability problem, the machine learning-guided screens have been adopted, substantially lowering the experimental resource and time spent in the course of optimization by reliably predicting the fitness of the full virtual library including, for example, 10⁵-10¹² variants from models trained with a small subset of empirical fitness data including, for example, 10³-10⁴ or even fewer variants, making in-silico screens successfully improving activities as well as diversifying sequences for antibodies, ion channels, and viral capsids.

Recent breakthroughs of the ML-driven protein design showed that generic evolutionary and structural constraints were transferable from other proteins to guide the search of high-fitness variants. Hence, accurate evaluations of the mutational effects on the variants in a library is a crucial element in reducing sampling size and achieving zero-shot or few-shot predictions. For example, the identification of high-likelihood mutations from multiple sequence alignments of homologues sequences resulted in efficient library design. Other methods include utilization of protein language models that extract rules from all proteins structures to infer mutational effects for a specific protein. These searches may further reduce the training data size to fewer than 100 variants, achieving low-N learning. Although these methods successfully shortlisted functional and better-than-wild type (WT) variants for experimental validations to accelerate the design process, a strategy that enables more robust and accurate discovery of top variants is needed.

The development of the genome editing proteins is an area in timely need for switching from rational design, directed evolution, and combinatorial mutagenesis, which either require extensive understanding of the protein structure or laborious selection and cloning for variant screening, to ML-driven engineering. Variants with enhanced specificity, higher activity, smaller size, as well as new functions are sought after for a broad range of biotechnological and biomedical applications. However, the optimization of CRISPR editing proteins, such as Cas9, poses additional engineering challenges because that most of the optimizations involved multiple (for example, 2-7) mutations spanning many potential engineering amino acid sites. Furthermore, comparative methods for variants design are limited because only a handful of protein structures are available for the Cas proteins. To reserve the expensive in-depth on-target and off-target experimental validations for top-performing variants, a better search regime is needed to tackle such a tremendous number of possible mutations present in a design library.

### SUMMARY OF THE INVENTION

Embodiments of the subject invention pertain to methods and systems for machine learning-based in-silico screen to search for high-performance genome editor variants.

In one aspect, the present invention provides a machine learning-based system using low-N learning to accurately identify variants for engineer genome editing proteins, which includes a CRISPR-associated protein mutational landscape, a zero-shot prediction module including a zero-shot predictor configured to predict the CRISPR-associated protein mutational landscape for a training dataset preparation, a machine learning-assisted directed evolution (MLDE) prediction module configured to perform predictions based on the training dataset, such that the identified variants are capable of being used for editing nucleic acids. The zero-shot predictor employs a clustered-based sampling (CS) or a Top regime method, and wild-type sequence is added in the training dataset. Multi-round sampling of CS/MLDE predictions are performed to identify and enrich the variants. The multi-round sampling comprises at least two rounds of sampling, and the wild-type sequence is added in a first-round sampling of the multi-round sampling.

In one embodiment, the zero-shot predictor is based on an arDCA method, an EVmutation method, an EvoEF method, or a MSA method.

In another embodiment, the variants are targeted to nucleic acids with a single guide RNA (sgRNA).

In one embodiment, the multi-round sampling may comprise 1, 2 or 4 round(s) of sampling.

In another embodiment, the zero-shot predictor further employs adaptive sampling (+AS).

In one embodiment, the performance of the zero-shot predictor is evaluated based on performance metrics including Spearman's correlation (rho) and/or top 5% overlaps (overlap_5).

When performing multi-round sampling of the CS/MLDE predictions, a plurality of high-fitness variants are included and the experimental validations of the high-fitness variants are prioritized for each additional round of MLDE prediction such that the training dataset is augmented and the MLDE prediction of the next round is improved.

In another embodiment, a fully saturated library is built for extending cross-validation of in-silico prediction and experimental data, including a plurality of variants over three amino acid positions including 888, 889, and 909 in SaCas9 WED domain (SaCas9-WED) for extending cross-validation of in-silico prediction and experimental data.

In one embodiment, the MLDE prediction is performed based on SpCas9, SaCas9_WED-PI, and SaCas9_WED datasets.

In one embodiment, the performance of final round of the MLDE predictions is evaluated based on enrichment, NDCG, rho, selectivity, and a number of top 50 variants correctly identified in the predictions.

In another aspect, the present invention provides a method to accurately identify variants for engineer genome editing proteins by a machine learning-based system using low-N learning, including performing zero-shot predictions by a zero-shot predictor together with a clustered-based sampling (CS) or a Top regime method with wild-type sequence being added for training data preparation; performing machine learning-assisted directed evolution (MLDE) predictions based on the training data; and performing multi-round sampling of the CS/MLDE predictions to identify and enrich the variants, such that the identified variants are capable of being used for nucleic acid editing. The multi-round sampling comprises at least two rounds of sampling. The wild-type sequence is added in a first-round sampling of the multi-round sampling.

In one embodiment, the zero-shot predictor is based on an arDCA method, an EVmutation method, an EvoEF method, or a MSA method.

In one embodiment, the identified variants are targeted to nucleic acids with a single guide RNA (sgRNA).

In one embodiment, the multi-round sampling may comprise 1-4 round(s) of sampling such as 1, 2, 3, or 4 round(s) of sampling.

In another embodiment, the method further including performing adaptive sampling (+AS).

In one embodiment, the performance of the zero-shot predictor is evaluated based on performance metrics including Spearman's correlation (rho) and/or top 5% overlaps (overlap_5).

In the step of performing multi-round sampling of the CS/MLDE predictions, a plurality of high-fitness variants are included and the experimental validations of the high-fitness variants are prioritized for each additional round of MLDE prediction such that the training data are augmented and the MLDE prediction of the next round is improved.

In another embodiment, the method further including building a fully saturated library for extending cross-validation of in-silico prediction and experimental data. The fully saturated library includes a plurality of variants over three amino acid positions, and wherein the three amino acid positions are 888, 889, and 909 in SaCas9 WED domain (SaCas9-WED).

In one embodiment, the MLDE prediction is performed based on SpCas9, SaCas9_WED-PI, and SaCas9_WED datasets.

In one embodiment, the performance of final round of the MLDE predictions is evaluated based on enrichment, NDCG, rho, selectivity, and a number of top 50 variants correctly identified in the predictions.

### BRIEF DESCRIPTION OF THE DRAWINGS

Embodiments of the invention are described in more details hereinafter with reference to the drawings, in which:

FIG. 1 shows the concept of the invention. Zero-shot predictors sample regions of the mutation landscape to generate screening library that are likely to be enriched with high performance variants for training. The subsequent multiple-round sampling guided by the machine learning-assisted directed evolution prediction module then enables the pick and validate process for identification of the top variants.

FIGS. 2A-2C are plot diagrams showing that zero-shot predictors serve as useful guides on training data preparation for low-N prediction, wherein FIG. 2A shows performance of the zero-shot predictor applying to CRISPR-Cas datasets, wherein the two metrics, Spearman's correlation (rho) and the discovery rate of top 5% variants are calculated over 5 CRISPR-Cas mutagenesis screens, in the boxplots, the box indicates the quantiles and the middle bar indicates median; whiskers extends to 1.5*inter-quartile range, points indicate prediction performance on screen against each sgRNA in the dataset, black dashed line indicates the expected top 5% overlap from random sampling, bar charts show the frequency for the zero-shot predictor to be identified as the best predictor(s) in each screen against a sgRNA over the 5 CRISPR-Cas mutagenesis screen (total=12 sgRNA screens); and FIGS. 2B-2C show the improvement of machine learning-assisted directed evolution (MLDE) prediction when the zero-shot predictors and various sampling strategies are utilized together with the MLDE, wherein the bar charts show the relative improvement of selectivity and NDCG to random sampling of 12, 24, and 48 variants as low-N training data, four zero-shot predictors (EVmutation, EvoEF, MSA, and arDCA) are combined with five sampling strategies (direct-ranking, CS, CS+adaptive sampling, CS+WT, and CS+adaptive sampling+WT), the bar height indicates mean relative improvement and points indicate individual improvement from each sgRNA dataset: 2 sgRNAs of SpCas9 and 3 sgRNAs of SaCas9_WED-PI, 0 indicates the same MLDE performance as random sampling, black dashed line indicates the relative improvement of MLDE using 20% of the library as the training data, according to an embodiment of the subject invention;

FIGS. 3A-3C show that iterative sampling improves low-N prediction accuracy, selectivity, and the number of top variants identified, wherein FIG. 3A is a schematic representation showing the workflow of the iterative sampling for low-N prediction. In first-round sampling, the training data is prepared for MLDE either by using CS or by selecting the top variants from zero-shot prediction, after the first round sampling, the training data is prepared for the second round of MLDE by adding in variants either selected using CS or by their predicted fitness from MLDE, in a similar manner, as the second-round sampling, the third and the fourth rounds of sampling are performed followed by MLDE to predict the fitness of the library, the final round of MLDE is used for performance evaluation; wherein FIG. 3B shows comparison of the sampling strategies based on MLDE performance, the scatter plot shows NDCG (left panel) or Top 50 identified (right panel) against selectivity of MLDE performance based on a training dataset of 48 samples generated from a combination of the zero-shot predictor and the iterative sampling strategy, each point indicates the mean performance values of NDCG/Top 50 identified vs selectivity, summarized from MLDE runs over the combination of zero-shot predictors, the number of rounds of the iterative sampling, and the sampling strategy of the first round (CS, CS+AS, CS+WT vs CS+AS+WT), black dashed line indicates the performance of MLDE using 20% of the library as training data; and wherein FIG. 3C shows identification of the best iterative sampling strategies from arDCA and EVmutation zero-shot predictions of 4-round sampling, using top 50 identified and selectivity as the paramount metrics, the best sampling strategies for the top zero-shot predictors EVmutation and arDCA in the scatter plots are labelled, according to an embodiment of the subject invention;

FIG. 4 shows the amino-acid (AA) distance from WT of the top 5% predicted variants using different zero-shot predictors in the SpCas9 and SaCas9_WED-PI datasets, the box indicates the quantiles and the middle bar indicates median, whiskers extend to 1.5*inter-quartile range, and points indicates outliners, according to an embodiment of the subject invention;

FIG. 5 shows performance of MLDE prediction on SpCas9 using training datasets sampled based on different zero-shot predictors and different sampling strategies, wherein enrichment, NDCG, Spearman's rho, and selectivity are plotted, boxplots summarize the averaged values of 2 sgRNAs for the SpCas9 dataset when different training data sizes are used (12, 24, 48; left to right), the box indicates the quantiles and the middle bar indicates median; whiskers extend to 1.5*inter-quartile range, points indicate individual datasets of SpCas9 on 2 sgRNAs, and the dash line indicates the averaged MLDE performance using training data from randomly selected 20% of variants in the library, according to an embodiment of the subject invention;

FIG. 6 shows performance of MLDE prediction on SaCas9_WED-PI using training datasets sampled based on different zero-shot predictors and different sampling strategies, in which enrichment, NDCG, Spearman's rho, and selectivity are plotted, boxplots summarize the averaged values of 3 sgRNAs for the SaCas9_WED-PI dataset when different training data sizes are used (12, 24, 48; left to right), the box indicates the quantiles and the middle bar indicates median; whiskers extend to 1.5*inter-quartile range, points indicate individual datasets of SaCas9_WED-PI on 3 sgRNAs, and black dash line indicates the averaged MLDE performance using training data from randomly selected 20% of variants in the library, according to an embodiment of the subject invention;

FIG. 7 shows the average improvement of MLDE prediction by using various sampling strategies, wherein boxplots indicate the relative improvement on NDCG, enrichment, Spearman's rho, and selectivity to random sampling of 12, 24, and 48 variants as training data (points grouped from left to right), 0 indicates the same MLDE performance as random sampling, significant improvements of CS+WT over direct-ranking on NDCG and rho, and marginal improvement on enrichment (paired t-test p-values: NDCG-0.0028, rho-0.00071 and enrichment-0.051) are observed, no significant added benefits of adaptive sampling (+AS) over CS+WT (paired t-test p-values: NDCG-0.33, enrichment-0.34, rho-0.84, selectivity-0.39) are detected, the box indicates the quantiles and the middle bar indicates median, whiskers extend to 1.5*inter-quartile range, points indicate individual MLDE runs from combinations of training data size, zero-shot predictors, sampling strategies for each sgRNA, zero-shot predictors including EVmutation, EvoEF, MSA, and arDCA, and applying the zero-shot predictors such as EVmutation, EvoEF, MSA, and arDCA together with different sampling strategies including direct-ranking using the top variants predicted from the zero-shot predictor directly, cluster-based sampling (CS), CS with adaptive sampling (AS) and/or with wild-type (WT), and random sampling, and sgRNA datasets including 2 sgRNA of SpCas9 and 3 sgRNA of SaCas9_WED-PI are used, according to an embodiment of the subject invention;

FIG. 8 shows performance of MLDE prediction on SpCas9 using training datasets sampled based on different zero-shot predictors and iterative sampling strategies with training data size of 48, wherein enrichment, NDCG, Spearman's rho, selectivity, and the number of top 50 variants identified are plotted, boxplots summarize the averaged values of 2 sgRNAs for the SpCas9 dataset when different sampling strategies are used (1-round, 2-round, and 4-round sampling; left to right), the box indicates the quantiles and the middle bar indicates median, whiskers extend to 1.5*inter-quartile range, points indicate individual datasets of SpCas9 on 2 sgRNAs, and gray dash line indicates the averaged MLDE performance using training data from randomly selected 20% of variants in the library, according to an embodiment of the subject invention;

FIG. 9 shows performance of MLDE prediction on SaCas9_WED-PI using training datasets sampled based on different zero-shot predictors and iterative sampling strategies with training data size of 48, wherein enrichment, NDCG, Spearman's rho, selectivity, and the number of top 50 variants identified are plotted, boxplots summarize the averaged values of 3 sgRNAs for the SaCas9_WED-PI dataset when different sampling strategies are used (1-round, 2-round, and 4-round sampling; left to right), the box indicates the quantiles and the middle bar indicates median, whiskers extend to 1.5*inter-quartile range, points indicate individual datasets of SaCas9_WED-PI on 3 sgRNAs, and gray dash line indicates the averaged MLDE performance using training data from randomly selected 20% of variants in the library, according to an embodiment of the subject invention;

FIGS. 10A-10B show comparison between empirical and predicted fitness from low-N prediction on a fully saturated library of 8,000 variants over SaCas9's WED domain, wherein FIG. 10A shows scatter plots demonstrating predicted variants' fitness values from MLDE predictions using arDCA and EVmutation with cluster-based multi-round sampling CSTopCSTop and CSTopTopTop on 2 sgRNAs DTp56libD18 and DTp58lib21. The top-50 variants in DTp58libD21 are highlighted in black solid circles and top 50 variants in DTp56libD18 are outlined in gray solid circles. In the predictions, overlaps in top 50 variants between the predictions of DTp58libD21 and DTp56libD18 are labelled with light grey. Training data in DTp56libD18 prediction are outlined in black circles and that of DTp58libD21 are outlined in gray circles. The WT “NAL” prediction is indicated in the FIG. 10A; and wherein FIG. 10B shows scatter plot demonstrating the empirical fitness values (E-score) of DTp56libD18 and DTp58lib21, the top 50 variants in DTp58libD21 are highlighted in black and top 50 variants in DTp56libD18 are outlined in circles, and top variants harboring the N888G mutation are labeled, according to an embodiment of the subject invention;

FIG. 11 shows that N888G mutation increases the editing activity of SaCas9-KKH, wherein the editing activity is characterized by GFP disruption assays with three independent sgRNAs targeting sites with protospacer adjacent motif (PAM) accessible for KKH-SaCas9 (NNNRRT), wherein two of the three sgRNAs are the same ones used in the empirical saturation mutagenesis screen, the mean editing efficiency of the KKH-SaCas9 variant (n=3 biologically independent samples) is measured as the percentage of cells with depleted GFP fluorescence using flow cytometry. Mean efficiency is indicated as bar height and individual readings are plotted as points; and

FIGS. 12A-12B show benchmarking of a top variant identification pipeline (TopVIP) method in comparison with the existing methods, wherein FIG. 12A shows that the discovery rates of the top 1% variants in the library are plotted against selectivity for each dataset and FIG. 12B shows bar charts illustrating the number of times that a prediction method ranked as the best based on the following metrics: a number of top 50 variants identified (top_50), a number of top 1% variants identified, enrichment, selectivity, and NDCG for each dataset, according to an embodiment of the subject invention.

### DETAILED DESCRIPTION

In the following description, sampling strategies WT_CSTopCSTop and WT_CSTopTopTop (based on arDCA and/or EVmutation) are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Embodiments of the subject invention are directed to a machine learning-based top variant identification pipeline method and systems of genome editing for proteins such as Cas9 protein.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not prelude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

To obtain greatest number of best-performing variants with least amount of experimental effort over the vast combinatorial mutational landscape for boosting resource producibility for protein engineering, a simple and effective machine learning-based top variant identification pipeline (TopVIP) method is provided which outperforms the existing methods. The TopVIP method integrates zero-shot prediction and multi-round sampling to direct active learning via experimenting only a small number of predicted top variants. It is found that four rounds of low-N pick-and-validate sampling of 12 variants for machine learning yield the best accuracy of up to 92.6% in selecting the true top 1% variants in combinatorial mutant libraries. Alternatively, two rounds of 24 variants can also be used. Thus, the TopVIP method can successfully discover new protein variants from diverse families including the CRISPR-based genome editors, supporting its generalizable application for solving protein engineering tasks.

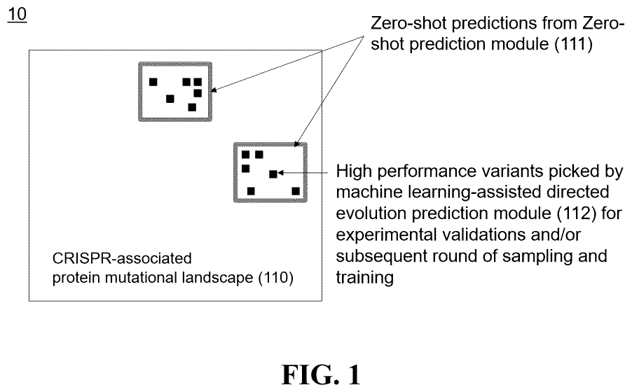

According to embodiments of the subject invention, a machine learning-based system is provided to accurately identify variants for engineer genome editing proteins (FIG. 1). The system (10) includes a CRISPR-associated protein mutational landscape (110), a zero-shot prediction module (111) including a zero-shot predictor configured to predict the CRISPR-associated protein mutational landscape for a training dataset preparation, a machine learning-assisted directed evolution (MLDE) prediction module (112) configured to perform predictions based on the training dataset, such that the identified variants are capable of being used for editing nucleic acids. The zero-shot predictor employs a clustered-based sampling (CS) or a Top regime method, and wild-type sequence is added in the training dataset. Multi-round sampling of CS/MLDE predictions are performed to identify and enrich the variants. The multi-round sampling comprises at least two rounds of sampling, and the wild-type sequence is added in a first-round sampling of the multi-round sampling.

According to other embodiments of the subject invention, a method is provided in which zero-shot predictors are coupled with clustered-based sampling (CS) for machine learning-assisted directed evolution (MLDE). First, the predictive accuracy of zero-shot predictors to the empirical fitness values of the CRISPR-Cas enzyme variants is determined. The zero-shot predictors are models that infer mutational effects based on indirect information such as homologous sequences or protein structural models. The zero-shot predictors that represent the state-of-art methods for mutational effect predictions are selected, including direct-coupling analyses (EVmutation and arDCA), a fine-tuned MSA transformer model csm_msa1_t12_100M_UR50S (MSA), a pre-trained NLP model (prot_bert-Naïve), and a structure-inspired predictor for protein stability change (EvoEF). The zero-shot predictions are performed on five sets of CRISPR-Cas library variants, with 12 sgRNAs tested in total, from published mutagenesis screens^{1, 9-12}, and the results of the predictions are compared with their empirical activities. Spearman's correlation (rho) and top 5% overlaps (overlap_5) are used as the performance metrics.

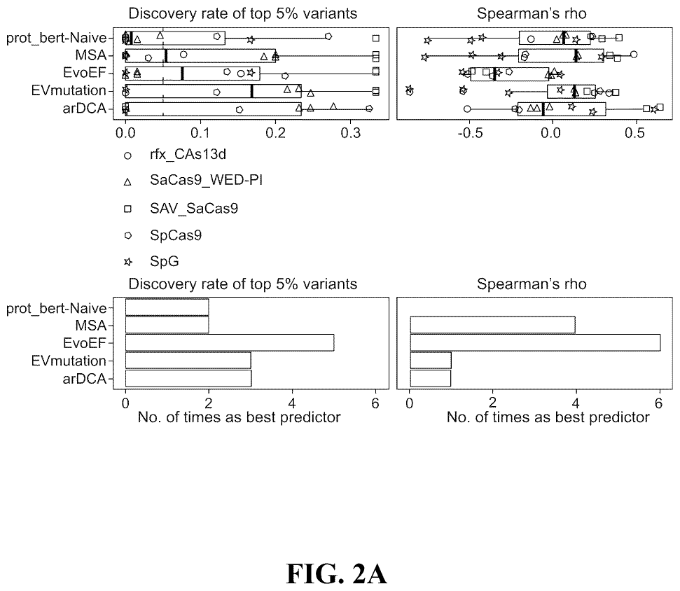

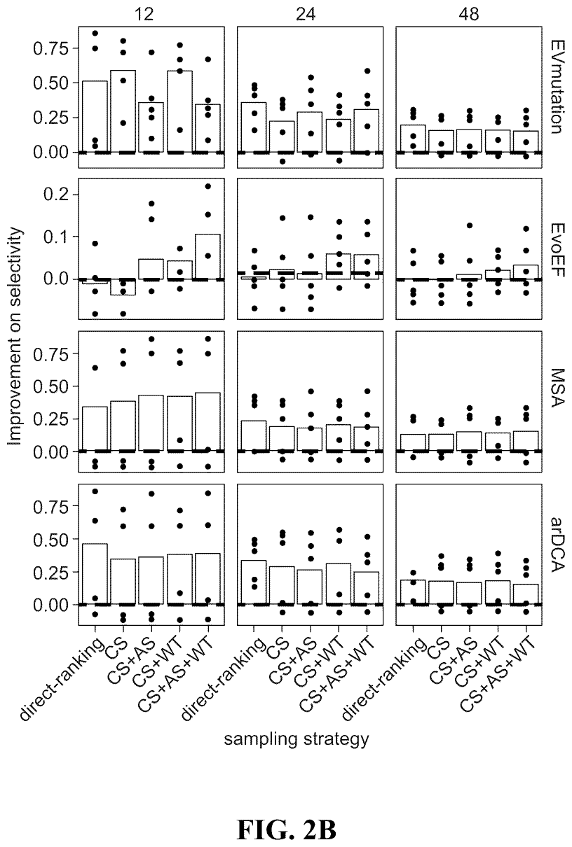

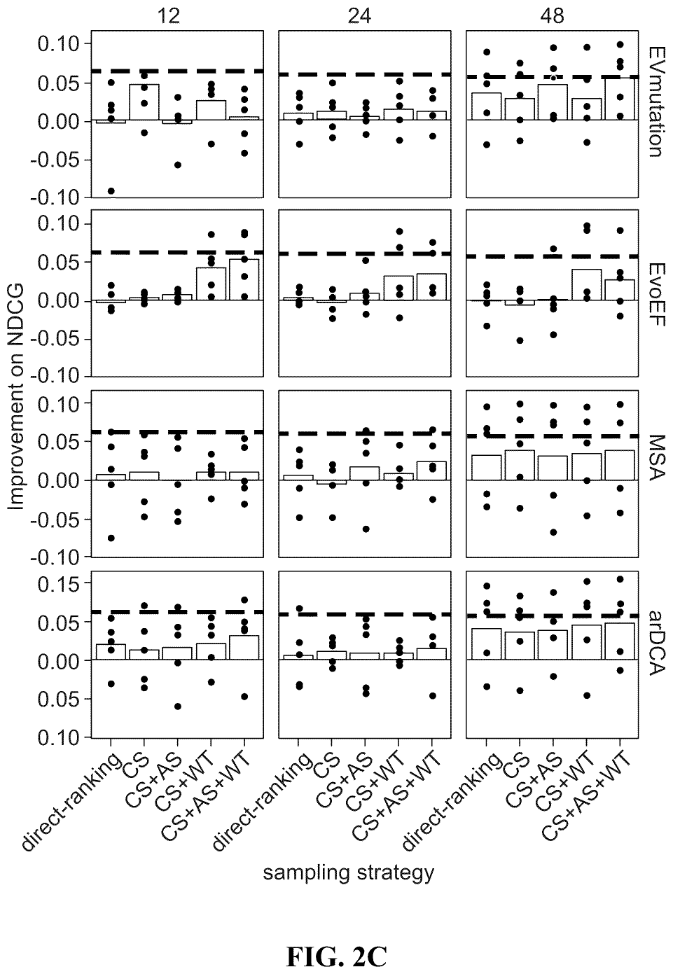

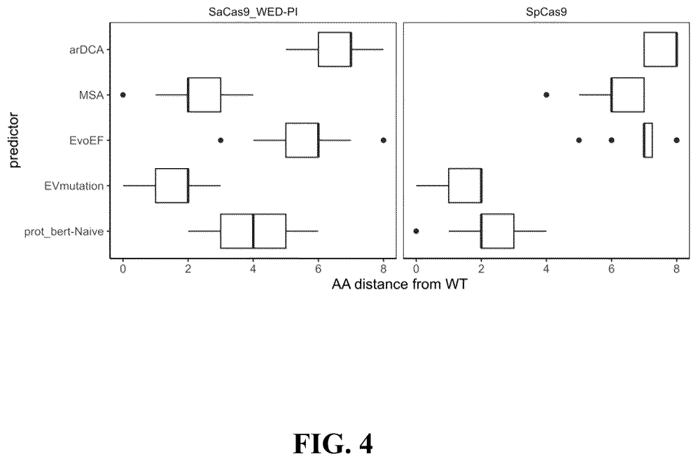

It is determined that all zero-shot predictors achieve modest correlations in most datasets (±0.1), encouraging enrichment for the top 5% overlap compared to the baseline of random sampling as shown in FIG. 2A. For each sgRNA, EvoEF is frequently identified as the best predictor for the Cas protein datasets, followed by EVmutation, arDCA, and MSA. Looking into the top 5% variants identified by different predictors in the SpCas9 and SaCas9_WED-PI libraries that experiment on larger numbers of variants, arDCA and EVmutation consistently identify the published top variants such as Opti-SpCas9⁹, SaCas9-N888Q, and SaCas9-A889S¹. It is noted that EVmutation exclusively predicts single and double mutants as top variants, while other predictors predict more multi-mutants as top variants as shown by the FIG. 4. Accordingly, although the zero-shot predictor alone is insufficient to recapitulate empirical fitness values, it can identify the top variants in certain cases that warrant downstream characterizations.

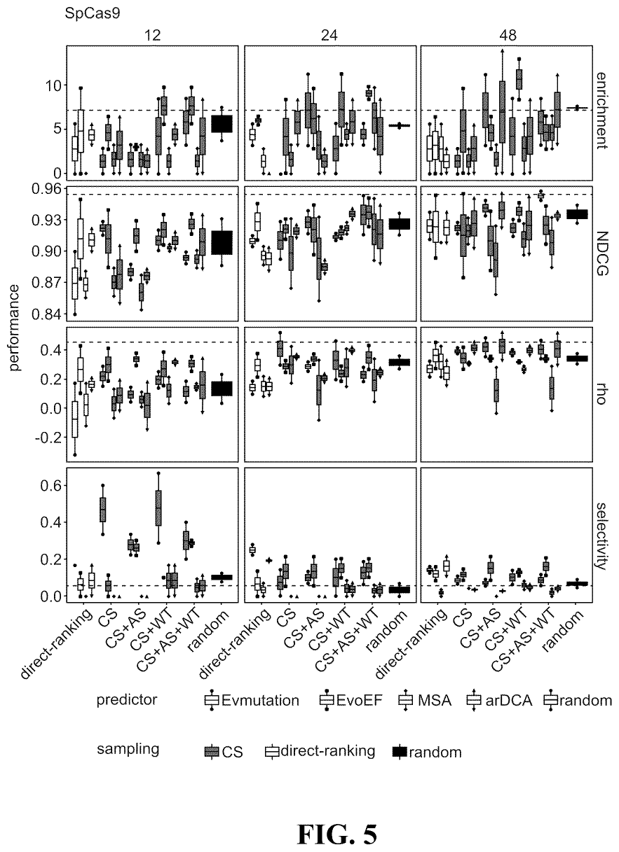

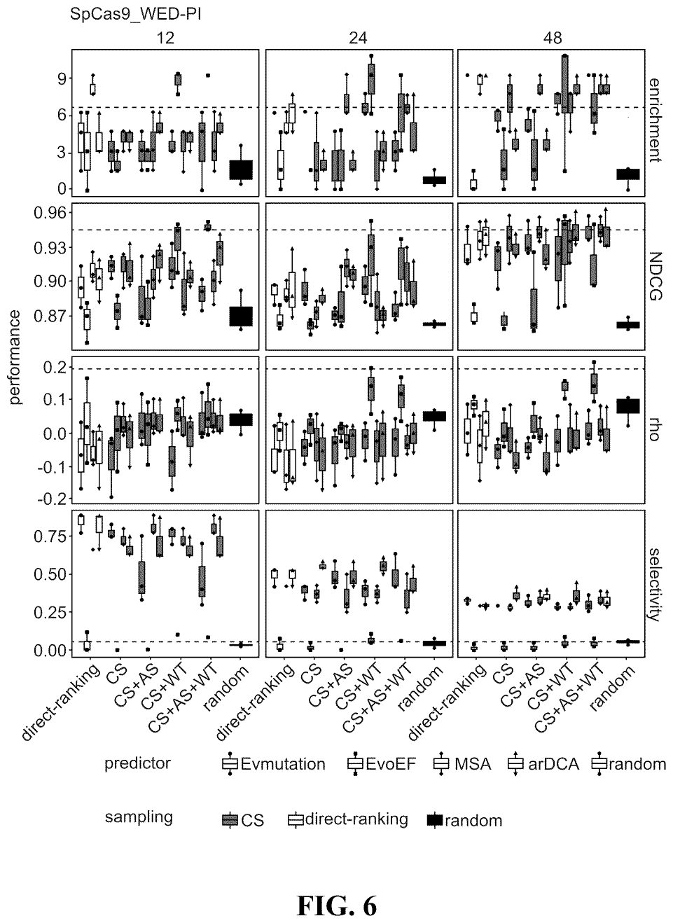

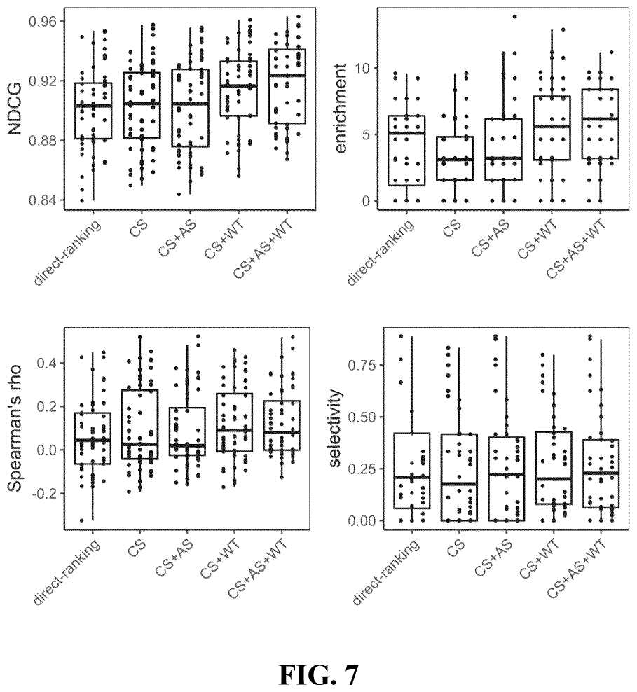

According to embodiments of the subject invention, the zero-shot predictions are useful as a guide for training data preparation to facilitate machine-learning based in-silico screen. In one embodiment, the SpCas9 and SaCas9_WED-PI datasets (with 952 and 1,296 variants, respectively) are employed, generating small training sets (low-N) of 12 (<1.3%), 24 (<2.5%), 48 (<5%) variants for the MLDE prediction. The zero-shot predictor such as EvoEF, EVmutation, arDCA, or MSA is applied with direct-ranking using the top variants directly predicted from zero-shot predictor, cluster-based sampling (CS), CS with adaptive sampling (AS) and/or with wild-type (WT), and random sampling. The performance of MLDE is evaluated using enrichment, rho, and discounted cumulative gain (NDCG) with a withheld test set of 20% of the library. To measure the level of prioritization on testing top variants, selectivity is derived for measuring the portion of top 5% library variants included in the training data. Remarkedly, sampling with cluster-based sampling with WT (CS+WT) shows the greatest improvement for most zero-shot predictors over random sampling (baseline) and the performance of MLDE is comparable with the published benchmark of using 20% of the library for the training data (indicated by dashed lines) as shown in FIGS. 2B, 2C, 5 and 6. There is no significant additional benefit of adaptive sampling (+AS) to CS+WT sampling as shown by FIG. 6, although this procedure may be beneficial for other protein optimization investigations.

Taken together, informative training datasets are derived for MLDE prediction by utilizing the zero-shot predictor and the CS+WT sampling. As a result, MLDE outperforms the random sampling in low-N setting while experimental validations are redirected to high-performing variants.

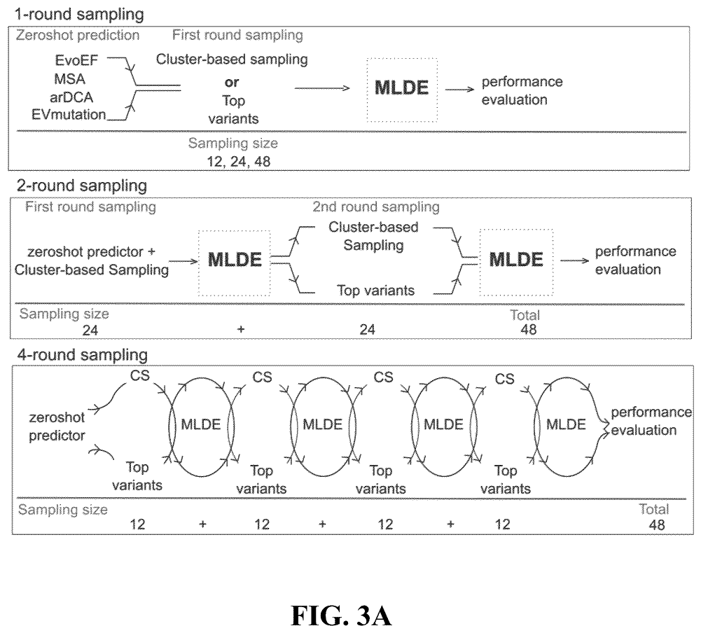

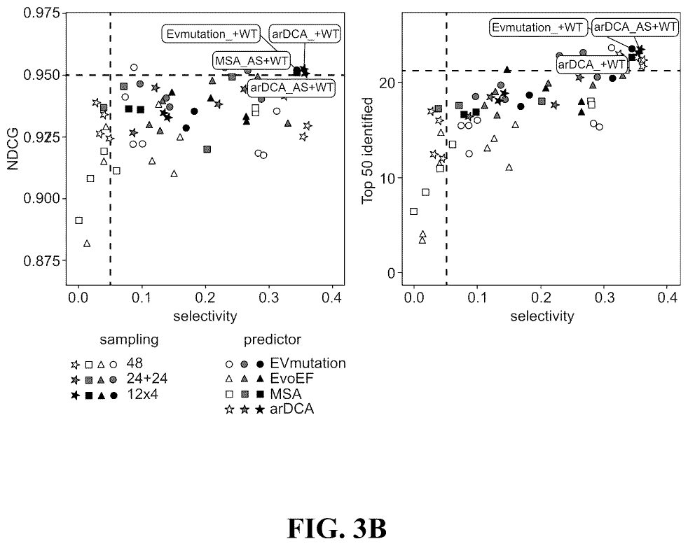

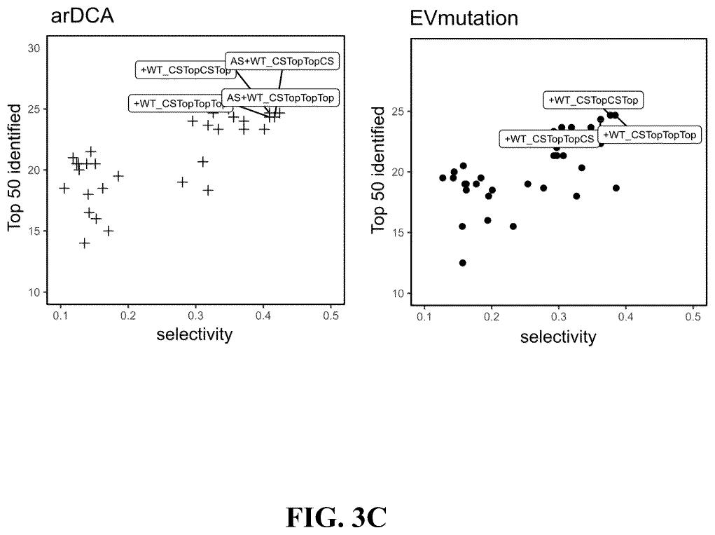

Moreover, when capping the training data size to 48 for low-N prediction, the method can further boost the MLDE performance and selectivity via multiple-round sampling. In one embodiment, 1-round 48, 2-round 24+24, and 4-round 12×4 sampling are performed as shown in FIG. 3A for training a dataset having 48 variants in total in the final MLDE run. In the 1-round sampling, 48 variants are only sampled once using the cluster-based sampling (CS) to simply picking the top variants (Top) based on zero-shot values and then MLDE is performed. In the 2-round sampling, 24 variants are first sampled using the CS or Top regime based on zero-shot values, next MLDE is performed, and then an additional 24 variants are sampled by the CS or the Top regime based on the MLDE prediction. Afterward, MLDE is performed again for the final prediction. There is a resultant of 4 combinations of CS/Top sampling regimes in 2-round sampling. Similarly, in the 4-round sampling, 12 variants arc sampled by the CS or the Top regime based on the zero-shot predictors to run the first round of MLDE, then 12 additional variants are sampled in each subsequent rounds by the CS or the Top regime based on the MLDE prediction of the previous round and then the MLDE is performed. The CS or Top sampling regime are cycled through for 4 rounds, resulting in 16 sampling combinations, and the final MLDE prediction is employed for performance evaluation.

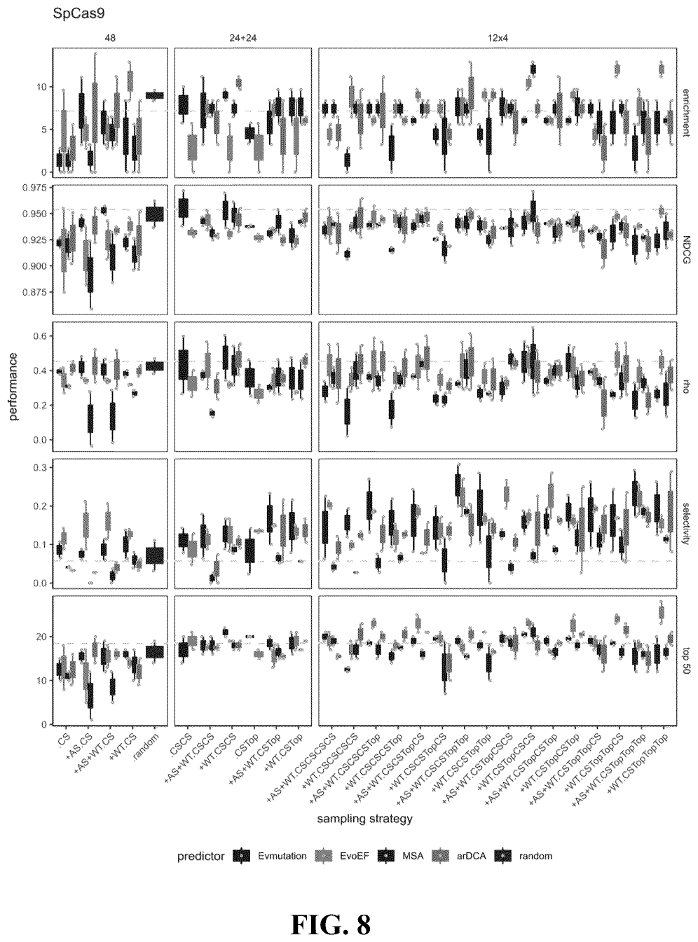

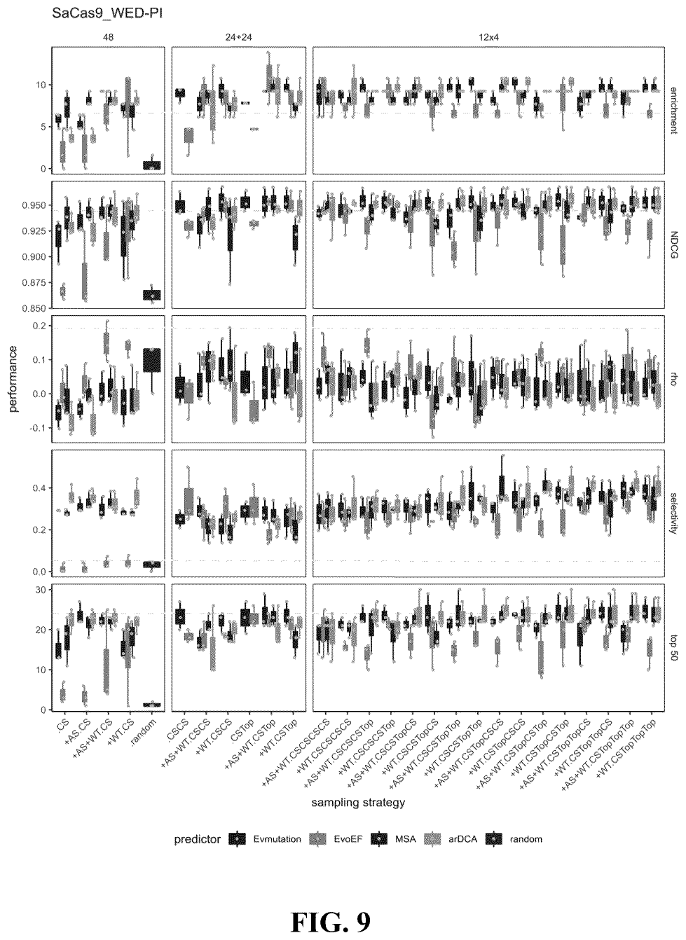

Each additional round of MLDE can augment the training data by including more high-fitness variants, prioritizing their experimental validation and improving the MLDE prediction of the next round. The best sampling method of zero-shot prediction CS+WT (and CS+AS+WT to confirm any added benefit of the setting of multiple-round sampling) is then applied to prepare the first round of training data for 2-round 24+24 and 4-round 12×4 sampling. In the subsequent round(s) of sampling, cither the top variants are picked from the prediction (Top) or the cluster-based sampling (CS) is applied to select variants to add to the training data. Performance of the final round of MLDE is evaluated regarding enrichment, NDCG, Spearman's rho, and selectivity. The number of top 50 variants identified correctly in the prediction is also counted to stringently evaluate MLDE's capability to find the top variants. It is found that the 4-round 12×4 sampling shows comparable NDCG and increased selectivity and top 50 identified compared to 2-round sampling and 1-round sampling as shown by FIGS. 8 and 9. Coupling with the augmented sampling strategies, NDCG and top50-identified are on par with the MLDE runs using 20% of the library as the training data. The improvement of selectivity from 1-round sampling to 4-round sampling is most significant when EvoEF is applied to the SaCas9_WED-PI dataset as shown by FIG. 9. In addition, using EVmutation and arDCA as zero-shot predictors with CS+WT and 4-round sampling yield the best results as shown in FIG. 3B. EVmutation and arDCA achieve the highest selectivity of 0.5 and 0.29 on SaCas9_WED-PI and SpCas9, respectively, generating about 10-fold and about 6-fold increases in selectivity compared to random sampling, highlighting the efficiency on experimental resource allocation of the method of embodiments of the subject invention. Among the best-performing predictors such as arDCA and EVmutation, on average, 8 out of the 18.4 (SpCas9) and 13 out of 21.2 (SaCas9_WED-PI) top 50 variants identified are validated as part of the training data, reaching a 16.7% and 27.1% efficiency on validating top variants with only 48 experiments, demonstrating remarkable improvement compared to 1-round (SpCas9: 10.42%, SaCas9_WED-PI: 19.8%) sampling and 2-round sampling (SpCas9: 14.8%, SaCas9_WED-PI: 18.9%). For EVmutation and arDCA, the best sampling strategies are determined to be +WT_CSTopCSTop and +WT_CSTopTopTop, respectively, as shown in FIG. 3C.

After establishing the above sampling strategy using the only two available large datasets of Cas9 variant libraries with focused mutations, that is, 2-3 selected amino acid residues per position, a fully saturated library which is a synthetic library expressed in mammalian cells and includes 8,000 variants over three amino acid positions (888, 889, and 909) in SaCas9 WED domain (SaCas9-WED) is built for extending the cross-validation of in-silico prediction and experimental data. The on-target activities of SaCas9-WED variants are characterized by two sgRNAs (DTp56libD18 and Dtp58libD21). The DCA and EVmutation zero-shot predictions are applied to the 8,000 SaCas9-WED variants and 48 experimental validations are selected via 4-round sampling with +WT_CSTopCSTop or +WT_CSTopTopTop. With the predictions from the combinations of predictors and sampling strategies, experimentally testing merely 48 variants (only 0.6% of variants from the SaCas9-WED library) results in validations of up to 28 top 5% variants in the library with a selectivity score of 0.64 (EVmutation), which is a 12.7-fold increase in the selectivity compared to the random sampling as shown in Table 1. Up to 22 top 50 variants are successfully identified with the method of embodiments of the subject invention and 13 of them are already validated in the training data as shown in Table 1.

Table 1 below shows performance of the MLDE predictions on a fully saturated mutagenesis library of SaCas9 using different zero-shot predictors and sampling strategies, according to an embodiment of the subject invention, wherein the fully saturated mutagenesis library comprises 8,000 variants over three amino acid positions (888, 889, and 909) in SaCas9's WED domain (SaCas9-WED). Two sgRNAs (DTp56libD18 and DTp58lib21) are used to evaluate the editing efficiency of the library variants, and the number of samples in training data omits variants with missing fitness values.

TABLE 1

| | Top 5% | No. of | | | # Top 50 |
|---|---|---|---|---|---|
| Sample (sgRNA_zero-shot | variants | samples | | Top 50 | variants in |
| predictor_sampling | in training | in training | | variants | training |
| method) | data | data | Selectivity | identified | data |
| DTp56libD18_EVmutation_CSTopCSTop | 28 | 44 | 0.64 | 21 | 13 |
| DTp56libD18_EVmutation_CSTopTopTop | 25 | 44 | 0.57 | 14 | 11 |
| DTp56libD18_arDCA_CSTopCSTop | 23 | 46 | 0.50 | 22 | 13 |
| DTp56libD18_arDCA_CSTopTopTop | 20 | 46 | 0.43 | 19 | 8 |
| DTp58libD21_EVmutation_CSTopCSTop | 20 | 44 | 0.45 | 9 | 4 |
| DTp58libD21_EVmutation_CSTopTopTop | 20 | 44 | 0.45 | 5 | 4 |
| DTp58libD21_arDCA_CSTopCSTop | 18 | 46 | 0.39 | 9 | 2 |
| DTp58libD21_arDCA_CSTopTopTop | 9 | 46 | 0.20 | 4 | 2 |

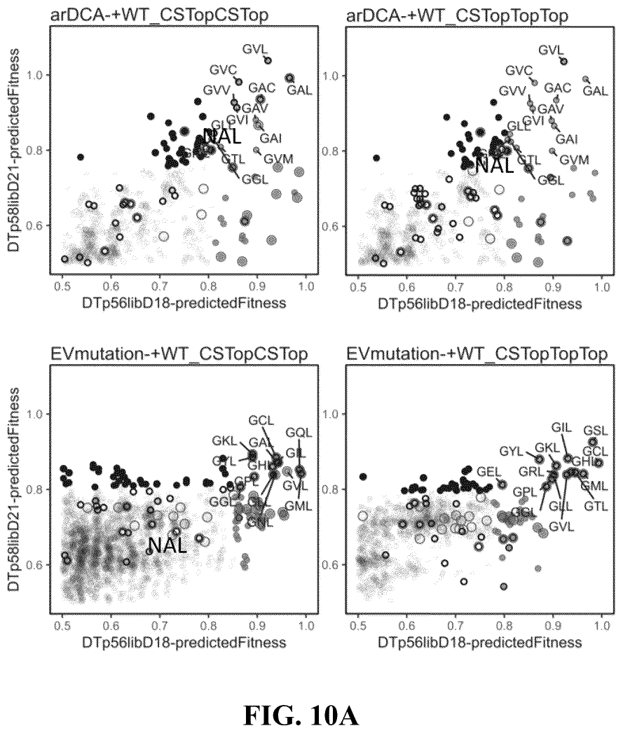

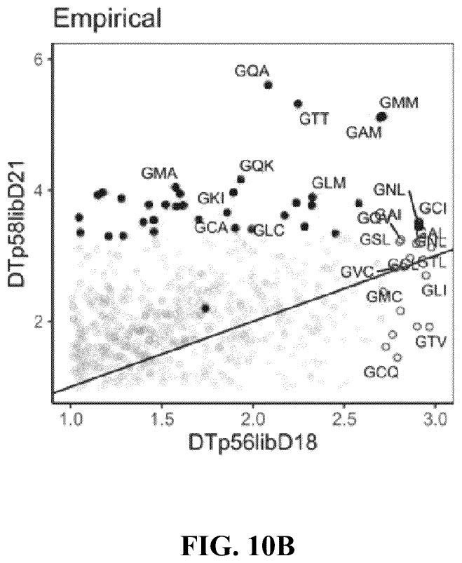

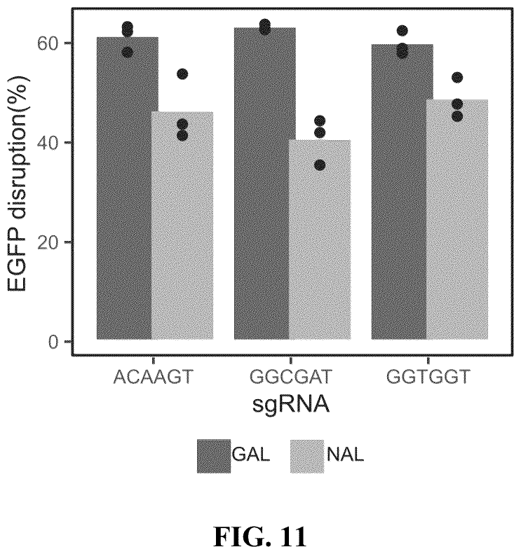

The MLDE predictions converge on the beneficial mutation N888G as shown by FIG. 10A, aligning with the empirical screen data as shown by FIG. 10B. Thus, it is confirmed that the N888G mutation increases SaCas9's editing activity in individual GFP disruption assays, which is shown by FIG. 11. According, the low-N iterative ML-based top variant identification pipeline (TopVIP) greatly reduces experimental burdens on screening and validation in the search of the top-performing variants.

### Comparison to Existing Sampling Strategies

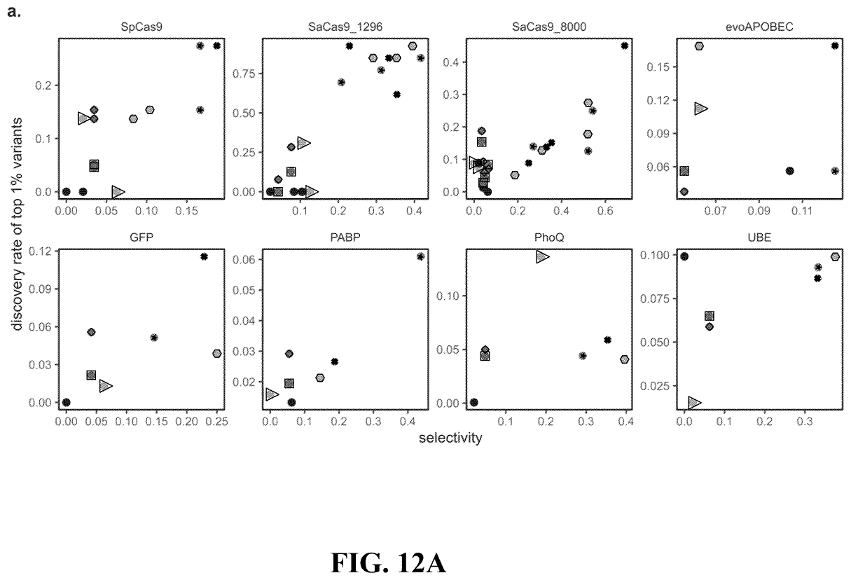

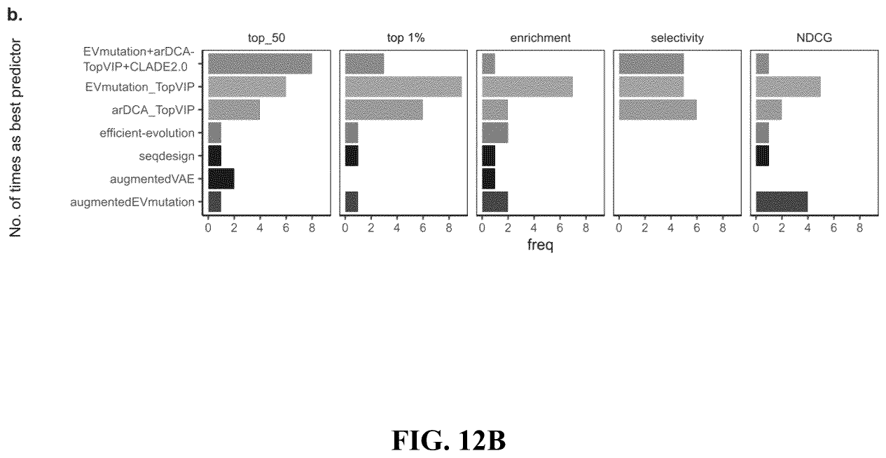

To investigate the scope of general applications of the pipeline, the TopVIP workflow is applied to four published mutagenesis datasets that contain at least 90% muti-mutants in the library. Meanwhile, the method of the embodiment of the subject invention is compared with the existing machine-learning models or existing sampling strategies for protein optimization under the low-N setting of 48 training datapoints. First, four more multi-mutant mutagenesis datasets (˜90% of the variants contain at least 2 mutations from the wild-type) are included on GFP, PABP, UBE and PhoQ with a library size ranging from 7,000-160,000 in addition to the previously published Cas9 datasets, the in-house Cas9 and cvoAPOBEC mutagenesis screen. It is found that the method of the embodiment of the subject invention in general outperforms the autoregressive model, the augmented representations, and the efficient-evolution method on selectivity and the discovery of top variants as shown by FIG. 12A and slightly better performance compared to NDCG. On average, the TopVIP method achieves a discovery rate of 0.277 for top 1% variants (arDCA_TopVIP: 0.279; EVmutation_TopVIP: 0.293; EVmutation+arDCA_TopVIP+CLADE2.0:0.258); it is 4.6-fold higher than comparing to that of the existing methods (augmentedEVmutation: 0.092; augmentedVAE: 0.053; seqdesign: 0.021; efficient-evolution: 0.075). In SaCas9 optimization tasks, the TopVIP method discoveres an average of 10.6 (arDCA_TopVIP: 11-12; EVmutation_TopVIP: 8-12; EVmutation+arDCA_TopVIP+CLADE2.0:9-11), compared to 0.86 (augmentedEVmutation: 0-4; augmentedVAE: 0-2; seqdesign: 0; efficient-evolution: 0-4), out of the 13 top 1% variants, reaching a 12.3-fold greater and up to 92.6% prediction accuracy on SaCas9_WED-PI dataset. The TopVIP method achieves an average selectivity of 0.39 (arDCA_TopVIP: 0.19-0.52; EVmutation_TopVIP: 0.25-0.69; EVmutation+arDCA_TopVIP+CLADE2.0:0.19-0.54), compared to 0.04 (augmentedEVmutation: 0.03-0.06; augmentedVAE: 0.03-0.06; seqdesign: 0.02-0.06; efficient-evolution: 0-0.04), achieving a 10.0-fold increase and up to a 68.8% selectivity in optimizing the SaCas9 WED. The TopVIP ranks top across datasets, demonstrating its robust performance for different proteins as shown in FIG. 12B. It is also found that there are negligible added benefits in combining zero-shot arDCA and EVmutation features using CLADE 2.0 for the first-round sampling in the TopVIP.

In summary, the zero-shot predictors are proven to be advantageous for enriching training data with functional variants and the benefit is twofold: prioritizing experimental characterization on high-fitness variants and improving the performance of MLDE. It is also found that applying CS to the zero-shot predictors and adding WT (that is, CS+WT) to the training set diversify the training set while keeping the functional variants. WT is almost always functional in Cas protein optimization and thus including WT in the training set improves the performance of MLDE. Moreover, the multi-round sampling plays a crucial role in the effective searches for top variants for greatly reducing the screening burden. In the embodiment of the subject invention, the active learning method compensates the drawbacks of a small training data size and the inaccuracy of the zero-shot predictions. The improvement in 2-round sampling is evident compared to 1-round sampling, while improvement from 2-round to 4-round sampling are more prominent with respect to the selectivity and the number of top 50 variants identified. The optimal iterative sampling strategies by mixing CS with sampling top variants (Top) show that there is a trade-off between diversifying the dataset and validating the top predicted variants. Contrary to ftMLDE³ that aims at purging non-functional variants in the training set, iterative sampling method maximizes the chance to include the top variants in the training set to boost the performance of MLDE, providing corollary benefits for prioritizing the experimental validation of the top variants.

The low-N sampling strategy according to the embodiments of the subject invention provides a solution to Cas protein engineering, bypassing the laborious screening and in-depth studies of the protein structures. It is further proved that the workflow is useful for the optimization of the SaCas9 WED domain, reducing the experimental burden to 0.6%. Further mutagenesis screens may expand the scope of application of the workflow to the broad family of Cas enzymes. Additional relief of experimental burden may be achieved by developing meaningful protein representation and more accurate zero-shot predictions with better MSA. Augmenting the protein representation to capture the fitness landscape is an area of active research. The recent breakthrough includes ReLSO¹⁴ that jointly generates sequences and predicts fitness. Homologous sequences of Cas proteins are abundant in metagenomic data^{15, 16} and could be a powerful resource for creating evolutionary-based zero-shot predictors.

### Materials and Methods

### Published Cas Protein Mutagenesis Screen Datasets

The on-target activities of the Cas protein variants from Choi et al 9 (SpCas9 on Sg5 and Sg8), Thean et al¹ (SaCas9_WED-PI on ACAAGT, GGTGGT, and TGGAGT), Yuen et al 11 (SAV_SaCas9 on ZRP7B and ZRP7G), Walton et al 10 (SpCas9 on NGAT, NGCC, NGGG and NGTA), and Tong et al 12 (Cas13d on EGFP) are collected.

### Zero-Shot Predictions

The multiple sequence alignments (MSA) is generated for SpCas9, and SaCas9 using the EVcoupling¹⁷ server (worldwide website: evcouplings.org/) with the bitscore threshold of 0.3 and the MSA of Cas13d orthologues is obtained from Wei et al 18. Based on the MSA, the DCA model is obtained from plmc (worldwide website: github.com/debbiemarkslab/plmc) and arDCA5 (https://github.com/pagnani/ArDCA.jl) to calculate the mutational effects of each variant with the default parameters. DDG stability of each variant is inferred using EvoEF⁸ (worldwide wesbsite: github.com/tommyhuangthu/EvoEF) with the default parameters. To generate the zero-shot prediction of Prot-bert (“prot_bert”)⁷ and MSA transformer⁶ (“esm_msal_t12_100M_UR50S”) with naive probability, the MLDE package (https://github.com/fhalab/MLDE) zero-shot prediction with default parameters and the MSA generated from EVcoupling for each protein are used. The generate_encoding.py is modified as described by Thean¹ (worldwide website: github.com/AWHKU/RunMLDE_SpCas9) to accommodate focused libraries from the default fully-saturated mutagenesis option.

It is expected that prot_bert-Naive, EVmutation, and MSA transformer show positive correlation whereas EvoEF DDG_stability shows negative correlation with the empirical data. Negative arDCA ΔE indicates beneficial mutations whereas positive ΔΕ predicts a deleterious mutation. Hence, it is expected that arDCA shows negative correlation with the empirical data. The accuracies of the zero-shot predictor for the empirical data and the prediction are evaluated using Spearman's correlation and top 5% overlaps. The Spearman's correlation indicates the overall accuracy of the zero-shot prediction while the top 5% overlap indicates how many of the top-performing variants could be recovered if only the top 5% of the variants based on zero-shot predictions are validated.

### Cluster-Based Sampling for Training Data

The cluster-based sampling method with zero-shot or MLDE predictions is based on the CLADE method. More specifically, the Georgiev protein representation is first generated by MLDE. K-means clustering is then performed with Scikit-learn 0.23.1 (https://jmlr.csail.mit.edu/papers/v12/pedregosalla.html) on the Georgiev protein representation of the whole library after finding the optimal number of clusters for each library based on the Within-Cluster Sum of Square (WCSS). The data are clustered by the numbers of clusters from 2 to 30 and the inertia of the K-mean clustering is plotted against the number of clusters. The plot is manually examined to find the “elbow” of the graph and determine the optimal number of clusters. 10 clusters are assigned for SpCas9 and SaCas9_WED-PI datasets and 25 clusters for fully saturated mutagenesis on SaCas9 position 888 889 and 909 in the WED domain (SaCas9_WED). Then 20 clusters are assigned for the evoAPOBEC dataset. Among the benchmarking datasets, 30 clusters are assigned for GFP, 27 clusters for PABP and UBE, and 19 clusters for PhoQ. Next, sampling probabilities of each cluster are assigned depending on the mean fitness prediction of the cluster. Given the training data size, the library is sampled and the number of variants is determined to sample for each cluster. Then, the top variants of each cluster are selected to curate the training dataset. To achieve that, the variants are ranked according to their zero-shot/MLDE prediction. To perform adaptive sampling (AS), further predictions of the fitness values and uncertainty are generated by Gaussian Process Regressor with an RBF kernel in scikit-learn (https://jmlr.csail.mit.edu/papers/v12/pedregosalla.html). More specifically, the Georgiev representation is employed as the features and the zero-shot prediction is employed as the response variables to fit the Gaussian Process Regressor (n_restarts==9) with a RBF kernel using the following parameters: length_scale=1.0, length_scale_bounds=(1e-2, 1e2). After fitting the model, the mean predicted fitness and the standard deviation (uncertainty) of each variant are retrieved. The final predicted value for adaptive sampling is the sum of the mean predicted fitness and the standard deviation*\sqrt{4}. Based on the upper confidence bound, the variants are ranked within a cluster from the highest predicted values to the lowest and the sampling of the high fitness-high uncertainty variants is prioritized. The training data generated is then fed to the MLDE for fitness prediction of the whole library. Variants selected by the clustered sampling but missing empirical fitness values in the screen are also included in the training sets. Later in the benchmarking session, variants without empirical fitness values are eliminated from the sampling process.

The CS method of the embodiments of the subject invention is different from the CLADE and CLADE 2.0 methods in the way to assign sampling probabilities of the clusters as well as the way of sampling variants within a cluster. In summary, the existing CLADE methods perform multiple rounds of samplings to select variants and fine-tune the sampling probabilities of the clusters in each iteration of variants sampling. Moreover, the existing CLADE methods use empirical fitness values in calculating sampling probabilities for clusters after the first round of sampling as well as ranking the variants. The CS method of the embodiments of the subject invention is confined to rank clusters and variants based on zero-shot/MLDE predictions to achieve low-N sampling, and no empirical fitness values are used. In practice, empirical fitness values are often unavailable when starting a new protein optimization task.

### Running CS for Training Data Selection

The Georgiev protein representation is generated and the parameters of MLDE³ is described in Thean¹.

Clustering_sampling.py is run in CLADE 2 (worldwide website: github.com/WeilabMSU/CLADE) for training set selection. In brief, CS performs clustering on the Georgiev protein representation of the whole library and prioritizes sampling on the cluster enriched with high-fitness variants as well as the higher-fitness variants within a cluster. Sampling probabilities of a cluster depend on the mean fitness/mean fitness+uncertainty of the cluster, and variants are ranked according to their predicted/empirical fitness during the sampling. The training data generated by CS is then fed to MLDE for fitness prediction of the whole library. Variants selected by CS with missing fitness values in the screen are also included in the training set.

The number of clusters in optimal K-means clustering for SpCas9 and SaCas9 is determined by manual inspection of the elbow plot of WCSS (Within-Cluster Sum of Square) against the cluster number. 10 clusters are assigned for SpCas9 and SaCas9_WED-PI datasets and 25 clusters are assigned for fully saturated mutagenesis on SaCas9 position 888 889 and 909 in the WED domain (SaCas9_WED). Then, the clustering_sampling.py is modified such that sampling with or without adaptive sampling can be performed via Gaussian Process Regression. When CS sampling is performed without adaptive sampling, only K-mean clustering is performed, and sampling probabilities of a cluster are assign based on the mean fitness from either the zero-shot predictions or the MLDE predictions.

### Running and Evaluating MLDE

MLDE is run using the SpCas9, SaCas9_WED-PI and the SaCas9_WED datasets. First, the fitness (E-score) of the library is normalized via min-max normalization after removing the extreme values. Fitness values below −2 to −2 are assigned to SpCas9 data and fitness values below −2.5 to −2.5 are assigned to the SaCas9_WED-PI datasets, and the minimal and maximal fitness values are defined as the 1% and 99% of the variants. Next, 20% of the library is randomly selected and withheld with fitness values being recorded as the test set for the MLDE performance evaluation. Any variants in the test set are excluded even they are selected by CS, and they are replaced with the next variant in the rank by CS in the training data.

MLDE is run using the Georgiev encoding and an ensembled model of XGB linear-Tweedie, linearSVR, randomforest, and elasticNET with default parameters and 5 rounds of hyper-parameterizations. Using a fixed test dataset of 20% of the library having 190 randomly selected variants of the SpCas9 library and 260 of the SaCas9 WED-PI libraries, the sampling strategies are evaluated based on enrichment, Spearman's correlation, and discounted cumulative gain (NDCG) as described by Thean 1. The fraction of top 5% variants in the training data is defined as “selectivity” and the number of top 50 variants identified in the whole library by the prediction is defined as “Top 50 identified” to evaluate the capabilities of MLDE for identifying the top variants for downstream validations. It is observed that datasets generated from zero-shot prediction show smaller impacts on the SpCas9 library, in which the functional variants are abundant (Sg5: 143; Sg8: 11 variants with ≥70% of WT activity), when compared to the SaCas9_WED-PI screens (ACAAGT: 6; GGTGGT: 12; TGGAGT: 15 variants with ≥70% of WT activity), suggesting that the zero-shot predictors are particularly advantageous for preparing informative training datasets for the MLDE predictions when functional variants are rare in the library.

### Generation of Empirical Saturated Mutagenesis Screen Data on KKH-SaCas9

The in-house KHH-SaCas9 dataset including 8,000 variants are constructed and characterized. Saturated mutagenesis on 3 amino-acid positions (888, 889, and 909) over the WED domain is performed. The on-target activities of the KKH-SaCas9 variants are measured against two sgRNAs as the E-score derived from the high-throughput GFP disruption assay and deep sequencing as previously described 1. The two sgRNAs use target sites with protospacer adjacent motif (PAM) accessible for KKH-SaCas9, that is, DTp56libD18: GGCGAT as PAM, and DTp58libD21: GGTGGT as PAM. The amino acid sequence combo (AA Combo) is extracted from the deep sequencing data. Each unique 3-nucleotides patterns corresponding to amino acid positions 888, 889, and 909 are counted from read 1 and then the codons are translated to amino acid sequences to derive the AA Combo in python. The LFC of the AA Combo between the GFP+ and GFP− pools of FACS-sorted cells are calculated as the E-score by R programs.

### Validation of KKH-SaCas9's Editing Activity

KKH-SaCas9 variants are constructed with standard molecular cloning techniques, and the fluorescent protein disruption assay is performed as previously described.

### Data Pre-Processing, Data Analyses, Statistics Calculations, and Plotting

Python 3.7 is used for data pre-processing and R 4.1.2 is used for data analyses, statistical tests, and plotting with packages tidyverse 1.3.1, stringdist 0.9.8, ggplot2 3.3.6, readxl 1.4.1, openxlsx 4.2.5, ggrepel 0.9.1, ggsci 2.9, and patchwork 1.1.2.

### Code and Data Availabilities

The codes and material for zero-shot predictions and pre-processing screen data can be found at Figshare/github.

The ability to screen at larger scale with less experimental efforts can save time and cost in engineering diverse genome editors for applications. The in-silico method coupling zero-shot and multi-round sampling is established to guide curation of only a small informative experimental set within the large mutational space for training and then predict the top-performing variants at high selectivity and accuracy. With a reduced wet-lab screening burden by 96.3% and 99.4% for the focused and saturated combinatorial mutagenesis libraries of SaCas9, respectively, the method of the embodiment of the subject invention achieves up to 10.0-fold and 12.7-fold increases in the selection rates of top variants in comparison to random sampling, that is 50.0% and 63.6% of the top variants are experimentally characterized as part of the training set a priori. The method of the embodiments of the subject invention is validated for engineering other widely used genome editing enzymes such as SpCas9, providing an improved strategy and benchmark for accelerating CRISPR-Cas engineering.

In comparison with the existing CLADE and CLADEv2.0 methods that cluster the variants in the library based on representation features and require experimental data inputs, the method of embodiments of the subject invention adopts the zeroshot-predictor(s) as a fitness proxy for shortlisting variants in the first-round sampling and does not require any experimental data input (except in the case in which the experimental data input is added in the wild type as described), thereby providing performances superior to the CLADE and CLADEv2.0 methods.

According to the embodiments of the subject invention, the pick-and-validate method and system are provided for simultaneously achieving low-N learning and prioritizing the validation of the top variants in a multi-mutant library. The method is applied to the optimization of genome editing proteins, a diverse group of proteins that some members including the small-sized nucleases and base editors are only more extensively studied and compared recently due to their applicational values; and their homologous sequences and high-resolution crystal structures are scarce. The method of the embodiments of the subject invention focuses on discovery of top-performing variants via curating an informative training dataset for the machine learning-assisted directed evolution (MLDE) workflow to maximize resource efficiency of the screen. An informative training dataset typically fulfils two criteria: 1) contains functional variants, and 2) covers enough diversity to represent the whole library. The sampling method of the embodiments of the subject invention focuses on enriching top-1%-performing variants while maintaining the sequence diversity in the training data for accurate MLDE predictions. It is shown that the multi-round sampling is the key for enriching the high-fitness variants for experimental validations.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

REFERENCES: THE DISCLOSURES OF THE FOLLOWING REFERENCES ARE INCORPORATED BY REFERENCE
1. Thean, D. G. L. et al. Machine learning-coupled combinatorial mutagenesis enables resource-efficient engineering of CRISPR-Cas9 genome editor activities. Nat Commun 13, 2219 (2022).
2. Qiu, Y., Hu, J. & Wei, G. Cluster learning-assisted directed evolution. Nature Computational Science 1, 809-818 (2021).
3. Wittmann, B. J., Yue, Y. & Arnold, F. H. Informed training set design enables efficient machine learning-assisted directed protein evolution. Cell Syst (2021).
4. Hopf, T. A. et al. Mutation effects predicted from sequence co-variation. Nat Biotechnol 35, 128-135 (2017).
5. Trinquier, J., Uguzzoni, G., Pagnani, A., Zamponi, F. & Weigt, M. Efficient generative modeling of protein sequences using simple autoregressive models. Nat Commun 12, 5800 (2021).
6. Rao, R. et al. MSA Transformer. bioRxiv, 2021.2002.2012.430858 (2021).
7. Elnaggar, A. et al. ProtTrans: Toward Understanding the Language of Life Through Self-Supervised Learning. IEEE Trans Pattern Anal Mach Intell 44, 7112-7127 (2022).
8. Huang, X., Pearce, R. & Zhang, Y. EvoEF2: accurate and fast energy function for computational protein design. Bioinformatics 36, 1135-1142 (2020).
9. Choi, G. C. G. et al. Combinatorial mutagenesis en masse optimizes the genome editing activities of SpCas9. Nat Methods 16, 722-730 (2019).
10. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
11. Yuen, C. T. L. et al. High-fidelity KKH variant of Staphylococcus aureus Cas9 nucleases with improved base mismatch discrimination. Nucleic Acids Res (2022).
12. Tong, H. et al. High-fidelity Cas13 variants for targeted RNA degradation with minimal collateral effects. Nat Biotechnol (2022).
13. Hie, B. L. & Yang, K. K. Adaptive machine learning for protein engineering. Curr Opin Struct Biol 72, 145-152 (2022).
14. Castro, E. et al. Transformer-based protein generation with regularized latent space optimization. Nature Machine Intelligence 4, 840-851 (2022).
15. Yan, W. X. et al. Functionally diverse type V CRISPR-Cas systems. Science 363, 88-91 (2019).
16. Makarova, K. S. et al. Evolutionary classification of CRISPR-Cas systems: a burst of class 2 and derived variants. Nat Rev Microbiol 18, 67-83 (2020).
17. Hopf, T. A. et al. The EVcouplings Python framework for coevolutionary sequence analysis. Bioinformatics 35, 1582-1584 (2019).
18. Wei, J. et al. Deep learning and CRISPR-Cas13d ortholog discovery for optimized RNA targeting. bioRxiv, 2021.2009.2014.460134 (2022).

What is claimed is:

1. A machine learning-based system using low-N learning to accurately identify variants for engineer genome editing proteins, comprising:
a CRISPR-associated protein mutational landscape;
a zero-shot prediction module comprising a zero-shot predictor configured to predict the CRISPR-associated protein mutational landscape for a training dataset preparation, wherein the zero-shot predictor employs a clustered-based sampling (CS) or a Top regime method, and wild-type sequence is added in the training dataset;
a machine learning-assisted directed evolution (MLDE) prediction module configured to perform predictions based on the training dataset, such that the identified variants are capable of being used for editing nucleic acids, wherein multi-round sampling of CS/MLDE predictions are performed to identify and enrich the variants, wherein the multi-round sampling comprises at least two rounds of sampling, and the wild-type sequence is added in a first-round sampling of the multi-round sampling.

2. The machine learning-based system according to claim 1, wherein the zero-shot predictor is based on an arDCA method, an EVmutation method, an EvoEF method, or a MSA method.

3. The machine learning-based system according to claim 1, wherein the variants are targeted to nucleic acids with a single guide RNA (sgRNA).

4. The machine learning-based system according to claim 1, wherein the zero-shot predictor further employs adaptive sampling.

5. The machine learning-based system according to claim 1, performance of the zero-shot predictor is evaluated based on performance metrics including Spearman's correlation (rho) and/or top 5% overlaps (overlap_5).

6. The machine learning-based system according to claim 1, wherein, when performing multi-round sampling of the CS/MLDE predictions, a plurality of high-fitness variants are included and experimental validations of the high-fitness variants are prioritized for each additional round of MLDE prediction such that the training dataset is augmented and the MLDE prediction of the next round is improved.

7. The machine learning-based system according to claim 1, the fully saturated library is built for extending cross-validation of in-silico prediction and experimental data.

8. The machine learning-based system according to claim 7, wherein the fully saturated library comprises a plurality of variants over three amino acid positions, and wherein the three amino acid positions are 888, 889, and 909 in SaCas9 WED domain (SaCas9-WED).

9. The machine learning-based system according to claim 1, wherein the MLDE prediction is performed based on SpCas9, SaCas9_WED-PI, and SaCas9_WED datasets.

10. The machine learning-based system according to claim 1, wherein performance of final round of the MLDE predictions is evaluated based on enrichment, NDCG, rho, selectivity, and a number of top 50 variants correctly identified in the predictions.

11. A method to accurately identify variants for engineer genome editing proteins by a machine learning-based system using low-N learning, comprising:
performing zero-shot predictions by a zero-shot predictor together with a clustered-based sampling (CS) or a Top regime method with wild-type sequence being added for training data preparation:
performing machine learning-assisted directed evolution (MLDE) predictions based on the training data;
performing multi-round sampling of the CS/MLDE predictions to identify and enrich the variants, such that the identified variants are capable of being used for nucleic acid editing, wherein the multi-round sampling comprises at least two rounds of sampling, and the wild-type sequence is added in a first-round sampling of the multi-round sampling.

12. The method according to claim 11, wherein the zero-shot predictor is based on an arDCA method, an EVmutation method, an EvoEF method, or a MSA method.

13. The method according to claim 11, wherein the identified variants are targeted to nucleic acids with a single guide RNA (sgRNA).

14. The method according to claim 11, further comprising performing adaptive sampling.

15. The method according to claim 11, wherein performance of the zero-shot predictor is evaluated based on performance metrics including Spearman's correlation (rho) and/or top 5% overlaps (overlap_5).

16. The method according to claim 11, wherein, in the step of performing multi-round sampling of the CS/MLDE predictions, a plurality of high-fitness variants are included and the experimental validations of the high-fitness variants are prioritized for each additional round of MLDE prediction such that the training data are augmented and the MLDE prediction of the next round is improved.

17. The method according to claim 11, further comprising building a fully saturated library for extending cross-validation of in-silico prediction and experimental data.

18. The method according to claim 17, wherein the fully saturated library comprises a plurality of variants over three amino acid positions, and wherein the three amino acid positions are 888, 889, and 909 in SaCas9 WED domain (SaCas9-WED).

19. The method according to claim 11, wherein the MLDE prediction is performed based on SpCas9, SaCas9_WED-PI, and SaCas9_WED datasets.

20. The method according to claim 11, wherein performance of final round of the MLDE predictions is evaluated based on enrichment, NDCG, rho, selectivity, and a number of top 50 variants correctly identified in the predictions.
